(19) United States
(12) Patent Application Publication
(10) Pub. No.: US 2008/0147122 A1
(43) Pub. Date: Jun. 19, 2008

(54) Dynamic stabilization connecting member with molded inner segment and surrounding external elastomer

(76) Inventor: Roger P. Jackson, Prairie Village, KS (US)

Correspondence Address: John C. McMahon, PO Box 30069, Kansas City, MO, 64112, US

(21) Appl. No.: 12/070,535

(22) Filed: Feb. 19, 2008

Related U.S. Application Data
(63) Continuation-in-part of application No. 12/008,067, filed on Jan. 8, 2008.
Application No. 12/008,067 is a continuation-in-part of application No. 11/894,001, filed on Aug. 17, 2007.
(60) Provisional application No. 60/902,470, filed on Feb. 21, 2007.
(60) Provisional application No. 60/897,723, filed on Jan. 26, 2007.
(60) Provisional application No. 60/851,353, filed on Oct. 12, 2006.

(51) Int. Cl. A61B 17/58

(52) U.S. Cl. 606/246; 606/264; 606/278

(57) ABSTRACT

A dynamic fixation medical implant having at least two bone anchors includes a longitudinal connecting member assembly having an inner elastic molded core and an outer spacer, the core and spacer being disposed between a pair of solid substantially rigid end portions. The assembly may further include a washer and a nut with the inner core or core portion being pre-tensioned. Alternatively, the outer spacer is molded over the inner elastic core, the core being in a neutral, compressed, tensioned or bent orientation during over molding of the spacer.

### CROSS REFERENCE TO RELATED APPLICATIONS

This application claims the benefit of U.S. Provisional Application No. 60/902,470 filed Feb. 21, 2007, which is incorporated by reference herein. This application is also a continuation-in-part of U.S. patent application Ser. No. 12/008,067 filed Jan. 8, 2008 that claims the benefit of U.S. Provisional Application No. 60/897,723 filed Jan. 26, 2007, both of which are incorporated by reference herein. Further, this application is also a continuation-in-part of U.S. patent application Ser. No. 11/894,001 filed Aug. 17, 2007 that claims the benefit of U.S. Provisional Application No. 60/851,353 filed Oct. 12, 2006, both of which are incorporated by reference herein.

### BACKGROUND OF THE INVENTION

The present invention is directed to dynamic fixation assemblies for use in bone surgery, particularly spinal surgery, and in particular to longitudinal connecting members that cooperate with bone anchors or fasteners, the connecting members being attached to at least two bone anchors.

Historically, it has been common to fuse adjacent vertebrae that are placed in fixed relation by the installation therealong of bone screws or other bone anchors and cooperating longitudinal connecting members or other elongate members. Fusion results in the permanent immobilization of one or more of the intervertebral joints. Because the anchoring of bone screws, hooks and other types of anchors directly to a vertebra can result in significant forces being placed on the vertebra, and such forces may ultimately result in the loosening of the bone screw or other anchor from the vertebra, fusion allows for the growth and development of a bone counterpart to the longitudinal connecting member that can maintain the spine in the desired position even if the implants ultimately fail or are removed. Because fusion has been a desired component of spinal stabilization procedures, longitudinal connecting members have been designed that are of a material, size and shape to largely resist flexure, extension, torsion, distraction and compression, and thus substantially immobilize the portion of the spine that is to be fused. Thus, longitudinal connecting members are typically uniform along an entire length thereof, and usually made from a single or integral piece of material having a uniform diameter or width of a size to provide substantially rigid support in all planes.

An alternative to fusion, which immobilizes at least a portion of the spine, and the use of more rigid longitudinal connecting members or other rigid structure has been a “soft” or “dynamic” stabilization approach in which a flexible loop-, S-, C- or U-shaped member or a coil-like and/or a spring-like member is utilized as an elastic longitudinal connecting member fixed between a pair of pedicle screws in an attempt to create, as much as possible, a normal loading pattern between the vertebrae in flexion, extension, distraction, compression, side bending and torsion. Another type of soft or dynamic system known in the art includes bone anchors connected by flexible cords or strands, typically made from a plastic material. Such a cord or strand may be threaded through cannulated spacers that are disposed between adjacent bone anchors when such a cord or strand is implanted, tensioned and attached to the bone anchors. The spacers typically span the distance between bone anchors, providing limits on the bending movement of the cord or strand and thus strengthening and supporting the overall system. Such cord or strand-type systems require specialized bone anchors and tooling for tensioning and holding the chord or strand in the bone anchors. Although flexible, the cords or strands utilized in such systems do not allow for elastic distraction of the system once implanted because the cord or strand must be stretched or pulled to maximum tension in order to provide a stable, supportive system.

The complex dynamic conditions associated with spinal movement create challenges for the design of elongate elastic longitudinal connecting members that exhibit an adequate fatigue strength to provide stabilization and protected motion of the spine, without fusion, and that allow for some natural movement of the portion of the spine being reinforced and supported by the elongate elastic or flexible connecting member. A further challenge are situations in which a portion or length of the spine requires a more rigid stabilization, possibly including fusion, while another portion or length may be better supported by a more dynamic system that allows for protective movement.

### SUMMARY OF THE INVENTION

Longitudinal connecting member assemblies according to the invention for use between at least two bone anchors provide dynamic, protected motion of the spine and may be extended to provide additional dynamic sections or more rigid support along an adjacent length of the spine, with fusion, if desired. A longitudinal connecting member assembly according to the invention has an elastic mid-section or core segment fixed at either end to substantially non-elastic or rigid solid segments, illustrated as rods, each having bone anchor fixation end portions. The elastic core is molded in the presence of the rigid segments, flows into apertures formed in such segments and adheres to such segments, thereby gripping the segments and forming a substantially integral or discrete elongate member for attachment with a bone anchor at either end. The elastic core is typically surrounded by a spacer that is also elastomeric. In one of the embodiments of the invention, one of the rigid segments includes a threaded portion. The illustrated assembly further includes a compression washer and a compression member illustrated as a nut mateable with the threaded portion. When threadably attached to the threaded portion of the rigid segment, the nut compresses the washer that in turn compresses against the outer spacer and also places a distractive force on the elastic core, placing such core in tension by pulling or distracting the rigid elongate segments away from one another, resulting in a dynamic pre-tensioning of the elastic core. The longitudinal connecting member assembly is dynamically loaded prior to being operatively attached to at least a pair of bone anchors along a patient's spine. The tensioned inner core and the compressed spacer cooperate dynamically, both features having some flexibility in bending also, with the outer spacer protecting and limiting flexing movement of the inner core. The spacer may include one or more grooves to aid in compression upon installation between the rigid elongate segments.

In another embodiment according to the invention a longitudinal connecting member includes an elastic inner core and a slitted compressible outer spacer. The inner core is molded with rigid elongate segments on either side thereof. The outer spacer is received over the core that is either twisted or stretched to place the core in tension. The elongate segments at either end of the spacer compress the spacer while placing a distractive force on the inner core. In the twisted core embodiment, the assembly is pinned, fixing the core in the desired twisted orientation. Embodiments according to the present invention advantageously allow for axial distraction and compression of the connecting member assembly, thus, for example, providing shock absorption.

Further embodiments according to the invention include a molded elastic inner core and an over-molded spacer. The inner core is first molded with rigid elongate segments on either side thereof. An outer spacer is then molded over the inner core. The outer spacer also may be molded over portions of the rigid segments located at either side of the inner core. The inner core may be neutral (not tensioned, compressed or bent), tensioned and/or bent during the molding of the outer elastic spacer there-around. The molded inner core and molded outer spacer may be of the same or different durometers.

A variety of embodiments according to the invention are possible. For example, a longitudinal connecting member may extend between three or more bone anchors with some or all of the sections that are located between bone anchors having elastic molded cores and pull or push-on, slitted or over-molded outer spacers. Alternatively, some of the sections may be of a more rigid construction and not include elastic cores or spacers.

### OBJECTS AND ADVANTAGES OF THE INVENTION

An object of the invention is to provide dynamic medical implant stabilization assemblies having longitudinal connecting members that include an inner core having a flexible portion that allows for some protected bending, torsion, compression and distraction of the assembly. Another object of the invention is to provide such an assembly having an elastic inner core and a surrounding elastic spacer wherein the inner core may be pre-tensioned and/or pre-bent and the spacer may be pulled or pushed on the core or molded over the core. A further object of the invention is to provide dynamic medical implant longitudinal connecting members that may be utilized with a variety of bone screws, hooks and other bone anchors. Another object of the invention is to provide a more rigid or solid connecting member portion or segment, if desired, such as a solid rod portion integral to the core having the flexible portion. Additionally, it is an object of the invention to provide a lightweight, reduced volume, low profile assembly including at least two bone anchors and a longitudinal connecting member therebetween. Furthermore, it is an object of the invention to provide apparatus and methods that are easy to use and especially adapted for the intended use thereof and wherein the apparatus are comparatively inexpensive to make and suitable for use.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an enlarged perspective view of a dynamic fixation connecting member assembly according to the invention including first and second rigid rod sections an elastic core (shown in phantom), a spacer and a compression nut.

FIG. 2 is an enlarged side elevational view of the assembly of FIG. 1 with portions broken away to show the detail thereof.

FIG. 3 is a reduced and exploded side elevational view of members of the assembly of FIG. 1 prior to molding with the elastic core (shown in phantom) including the first rod section with a buttress, the second rod section with a thread, the spacer, a washer and the nut.

FIG. 4 is an enlarged perspective view of the first rod section shown in FIG. 3.

FIG. 5 is an enlarged perspective view of the second rod section shown in FIG. 3.

FIG. 6 is an enlarged perspective view of the washer shown in FIG. 3.

FIG. 7 is an enlarged perspective view of the nut shown in FIG. 3.

FIG. 8 is an enlarged bottom plan view of the nut shown in FIG. 3.

FIG. 9 is an enlarged perspective view of the spacer shown in FIG. 3.

FIG. 10 is a reduced perspective and partially exploded view of the assembly of FIG. 1 shown with a pair of bone screws and cooperating closure tops.

FIG. 11 is an enlarged perspective view of a second embodiment of a dynamic fixation connecting member assembly according to the invention showing a pair of rigid rod sections and a spacer therebetween.

FIG. 12 is an enlarged side elevational view of the connecting member assembly of FIG. 11.

FIG. 13 is an enlarged side elevational view similar to FIG. 12 with portions broken away to show the detail thereof.

FIG. 14 is an enlarged perspective view of the spacer of FIG. 11.

FIG. 15 is an enlarged side elevational view showing the assembly of FIG. 11 prior to dynamic loading thereof.

FIG. 16 is an enlarged perspective view of a third embodiment of a dynamic fixation connecting member assembly according to the invention showing a pair of rigid rod sections and a spacer therebetween.

FIG. 17 is an enlarged side elevational view of the assembly of FIG. 16 with portions broken away to show the detail thereof.

FIG. 18 is an enlarged side elevational view showing the assembly of FIG. 16 prior to dynamic loading thereof.

FIG. 19 is an enlarged front elevational view of a fourth embodiment of a dynamic fixation connecting member assembly according to the invention including an inner elastic core, a pair of stop plates and an outer over-molded elastic spacer.

FIG. 20 is a reduced perspective view of the embodiment of FIG. 19 shown before molding of the outer spacer thereon.

FIG. 21 is an enlarged cross-sectional view taken along the line 21-21 of FIG. 19.

FIG. 22 is an enlarged front elevational view of a fifth embodiment of a dynamic fixation connecting member assembly according to the invention including an inner elastic core, a pair of stop plates and an outer over-molded elastic spacer and showing a bone screw in phantom.

FIG. 23 is an enlarged front elevational view, similar to FIG. 22, with portions broken away to show the detail thereof.

FIG. 24 is a cross-sectional view taken along the line 24-24 of FIG. 22.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the connecting member assemblies of the application and cooperating bone anchors in actual use.

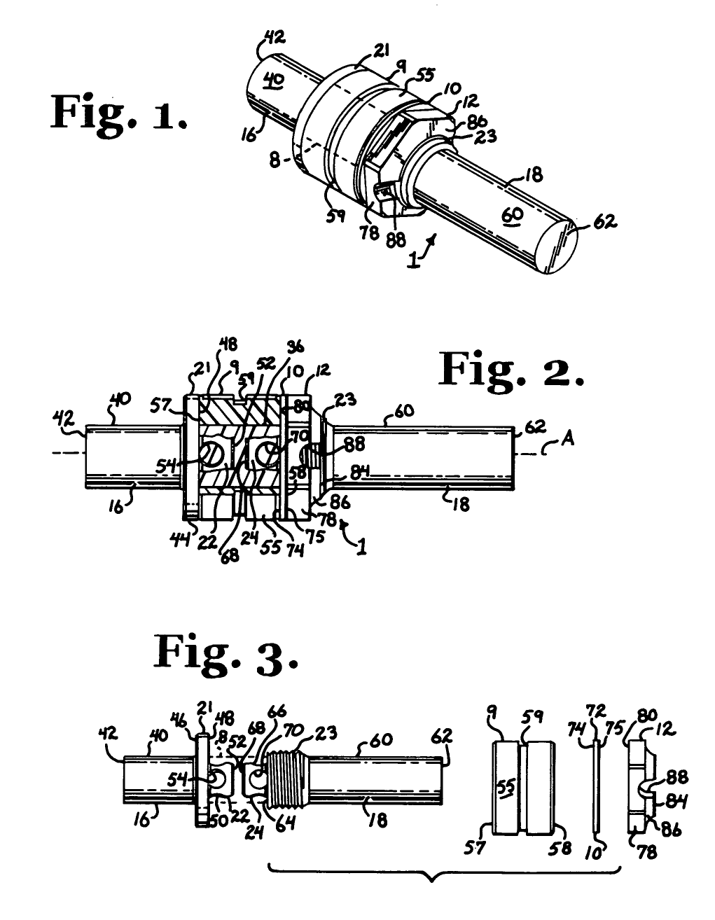

With reference to FIGS. 1-10, the reference numeral 1 generally designates a non-fusion dynamic stabilization longitudinal connecting member assembly according to the present invention. The connecting member assembly 1 is elongate and substantially cylindrical, having a central axis A. The assembly 1 includes a molded elastic substantially solid dynamic mid-portion or core 8, an elastic spacer 9, a washer 10 and a compression/distraction nut 12. The assembly 1 further includes elongate rigid segments 16 and 18 with the core 8 being disposed therebetween and attached to each of the segments 16 and 18. The segment 16 is substantially solid, rigid and cylindrical and further includes a buttress or plate 21 and a molding attachment member 22. The segment 18 is substantially solid, rigid and cylindrical, having a diameter the same or similar to a diameter of the segment 16. The segment 18 includes a threaded portion 23 and a molding attachment member 24 substantially similar or identical to the molding attachment member 22. The core 8 is fabricated from a molded elastomer, as will be described more fully below, in the presence of the segments 16 and 18, with molded plastic flowing through apertures of the attachment members 22 and 24 and thereafter adhering to such members as illustrated in FIG. 2.

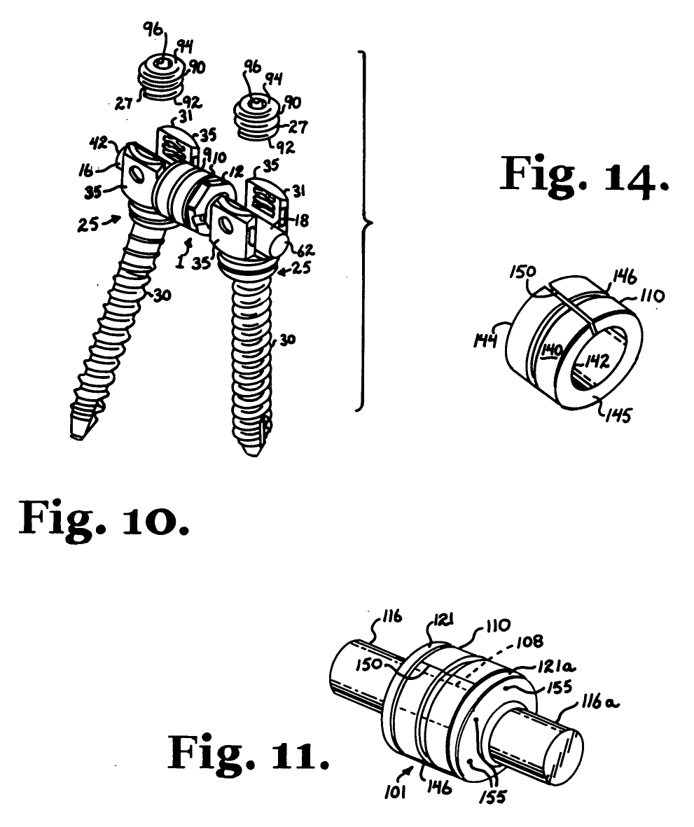

The washer 10 and the nut 12 are received by the segment 18 with an inner thread of the nut 12 mating with the outer threaded portion 23 as will be described more fully below. The dynamic connecting member assembly 1 cooperates with at least a pair of bone anchors, such as the polyaxial bone screws, generally 25 and cooperating closure structures 27 shown in FIG. 10, the assembly 1 being captured and fixed in place at the segments 16 and 18 by cooperation between the bone screws 25 and the closure structures 27. The dynamic core 8, that is pre-loaded and pre-tensioned with the spacer 9, washer 10 and cooperating nut 12, is disposed between the bone screws 25. It is foreseen that in some embodiments, the assembly 1 may include a small central lumen along an entire length thereof and opening at each end thereof to allow for threading therethrough and subsequent percutaneous implantation of the member 1.

Because the segments 16 and 18 are substantially solid and cylindrical, the connecting member assembly 1 may be used with a wide variety of bone anchors already available for cooperation with rigid rods including fixed, monoaxial bone screws, hinged bone screws, polyaxial bone screws, and bone hooks and the like, with or without compression inserts, that may in turn cooperate with a variety of closure structures having threads, flanges, or other structure for fixing the closure structure to the bone anchor, and may include other features, for example, break-off tops and inner set screws. It is foreseen that the substantially cylindrical core 8, segment 16, buttress 21 and segment 18 that are illustrated as having various circular cross-section may in other embodiments of the invention have other cross-sectional shapes, either along an entire length of the assembly 1 or portions thereof, including, but not limited to oval, square, rectangular and other curved or polygonal shapes. The bone anchors, closure structures and the connecting member assembly 1 are then operably incorporated in an overall spinal implant system for correcting degenerative conditions, deformities, injuries, or defects to the spinal column of a patient.

The illustrated polyaxial bone screws 25 each include a shank 30 for insertion into a vertebra (not shown), the shank 30 being pivotally attached to an open receiver or head 31. The shank 30 includes a threaded outer surface and may further include a central cannula or through-bore disposed along an axis of rotation of the shank to provide a passage through the shank interior for a length of wire or pin inserted into the vertebra prior to the insertion of the shank 30, the wire or pin providing a guide for insertion of the shank 30 into the vertebra. The receiver 31 has a pair of spaced and generally parallel arms 35 that form an open generally U-shaped channel therebetween that is open at distal ends of the arms 35. The arms 35 each include radially inward or interior surfaces that have a discontinuous guide and advancement structure mateable with cooperating structure on the closure structure 27. The guide and advancement structure may take a variety of forms including a partial helically wound flangeform, a buttress thread, a square thread, a reverse angle thread or other thread like or non-thread like helically wound advancement structure for operably guiding under rotation and advancing the closure structure 27 downward between the receiver arms 35 and having such a nature as to resist splaying of the arms 35 when the closure 27 is advanced into the U-shaped channel. For example, a flange form on the illustrated closure 27 and cooperating structure on the arms 35 is disclosed in Applicant's U.S. Pat. No. 6,726,689, which is incorporated herein by reference.

The shank 30 and the receiver 31 may be attached in a variety of ways. For example, a spline capture connection as described in U.S. Pat. No. 6,716,214, and incorporated by reference herein, is used for the embodiment disclosed herein. Polyaxial bone screws with other types of capture connections may also be used according to the invention, including but not limited to, threaded connections, frictional connections utilizing frusto-conical or polyhedral capture structures, integral top or downloadable shanks, and the like. Also, as indicated above, polyaxial and other bone screws for use with connecting members of the invention may have bone screw shanks that attach directly to the connecting member segment 16 or 18, or may include compression members or inserts that cooperate with the bone screw shank, receiver and closure structure to secure the connecting member assembly to the bone screw and/or fix the bone screw shank at a desired angle with respect to the bone screw receiver that holds the longitudinal connecting member assembly 1. It is foreseen that if the connecting member segments 16 and 18 are fabricated from a plastic such as polyetheretherketone (PEEK), it may be desirable to utilize bone screws that include both upper and lower compression inserts that have a saddle or U-shape configuration to closely engage such segments within the bone screw receiver. Although the closure structure 27 of the present invention is illustrated with the polyaxial bone screw 25 having an open receiver or head 31, it is also foreseen that a variety of closure structures may be used in conjunction with any type of medical implant having an open or closed head, including monoaxial bone screws, hinged bone screws, hooks and the like used in spinal surgery.

To provide a biologically active interface with the bone, the threaded shank 30 may be coated, perforated, made porous or otherwise treated. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃(PO₄)₂, tetra-calcium phosphate (Ca₄P₂O₉), amorphous calcium phosphate and hydroxyapatite (Ca₁₀(PO₄)₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

The longitudinal connecting member assembly 1 illustrated in FIGS. 1-10 is elongate, with the segments 16 and 18, the washer 10 and the nut 12 being made from metal, metal alloys or other suitable materials, including plastic polymers such as polyetheretherketone (PEEK), ultra-high-molecular weight-polyethylene (UHMWP), polyurethanes and composites. The molded elastic core 8 may be made of a variety of materials including plastics and composites. The illustrated core 8 is made from a plastic, such as a natural or synthetic elastomer or blend thereof, including, but not limited to polyisoprene (natural rubber), and synthetic polymers, copolymers, and thermoplastic elastomers, and mixtures thereof, with the illustrated core 8 being a polyurethane elastomer. With particular reference to FIG. 2, once molded, the illustrated core 8 is substantially solid, smooth and in the form of a cylinder having an outer surface 36 of uniform circular cross-section.

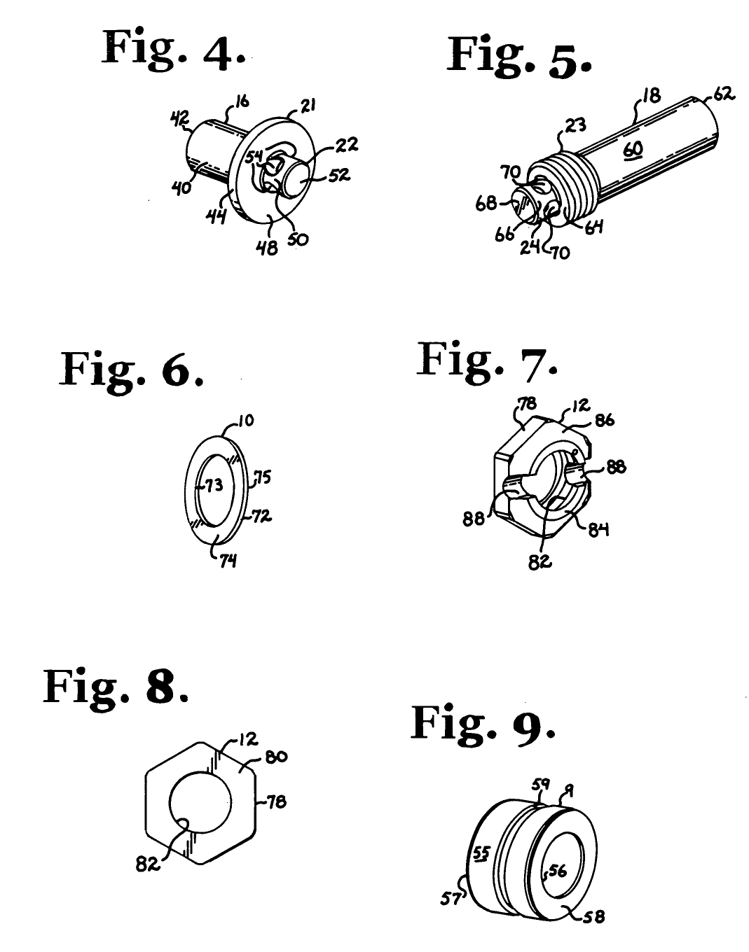

With particular reference to FIGS. 3 and 4, the rigid segment 16 has a circular cross-section with an outer substantially smooth cylindrical surface portion 40 extending from a planar end surface 42 to the integral buttress plate 21. The buttress plate 21 has an outer cylindrical surface 44, also of circular cross-section and having a diameter greater than a diameter of the cylindrical surface portion 40. The buttress plate 21 has opposed substantially planar surfaces 46 and 48. The surfaces 42, 46 and 48 are all disposed substantially perpendicular to the axis A. Extending from the surface 48 and along the axis A is the molding attachment member 22 that is integral with the plate 21. The member 22 has an outer curved surface 50 that is concave, circular in cross-section and extends from the surface 48 to an end surface 52 that is substantially perpendicular to the axis A. Formed in the surface 50 are a plurality of through bores 54. In the illustrated embodiment there are two through bores 54 that form a substantially hollow area within the member 22 to flow receive and provide for set-up and adherence to the plastic core 8. The plastic core 8 is molded adjacent to the plate 21 and thus also adheres to the surface 48 and the surface 52.

With particular reference to FIGS. 1-3 and 9, the sleeve or spacer 9 advantageously cooperates with the core 8, providing limitation and protection of movement of the core 8. The spacer 9 is substantially cylindrical and made from a plastic, such as a thermoplastic elastomer made from a polyurethane or polyurethane blend. The spacer 9 has an external substantially cylindrical surface 55 and an internal substantially cylindrical and smooth surface 56 defining a bore with a circular cross section extending through the spacer 9. The surfaces 55 and 56 extend between a pair of substantially planar end surfaces 57 and 58. When cooperating with the core 8 the end surfaces 57 and 58 are substantially perpendicular to the axis A. It is foreseen that in some embodiments, the spacer may be of square, rectangular or other cross-section including curved or polygonal shapes. In the illustrated embodiment, the spacer 9 further includes a compression groove 59. Spacers according to the invention may include one, none or any desired number of grooves. The illustrated groove 59 is substantially uniform and circular in cross-section, being formed in the external surface 55 and extending radially toward the internal surface 56. The internal surface 56 is of a slightly greater diameter than the outer diameter of the core 8 surface 36. The size of the internal surface 56 allows for axially directed sliding movement of the spacer 9 with respect to the core 8. As shown in FIG. 2, when the spacer 9 is initially placed on the core 8, the spacer 9 completely surrounds the core 8 and abuts against the buttress plate surface 48. The elastic core 8 and cooperating compressible spacer 9 allows the core 8 to twist or turn, providing some relief for torsional stresses. The spacer 9, however limits such torsional movement as well as bending movement, providing spinal support. Furthermore, because the spacer 9 is compressed during installation, the spacer and core 8 combination advantageously allow for some protected extension or distraction of both the core 8 and the spacer 9 as well as further compression of the assembly 1 at the core 8.

With particular reference to FIGS. 3 and 5, the rigid segment 18 has a circular cross-section with an outer substantially smooth cylindrical surface portion 60 extending from a planar end surface 62 to the integral threaded portion 23. The segments 16 and 18 have substantially the same diameter and each are sized and shaped to be received in the U-shaped channel formed between the arms 35 of a bone screw receiver 31 with the dynamic core 8 and spacer 9 combination being disposed between cooperating bone screws 25. The threaded portion or length 23 of the segment 18 has a minor or root thread diameter substantially the same or, as illustrated, slightly greater than a diameter of a remainder of the segment 18. The threaded portion 23 runs out at an end surface 64 that is substantially perpendicular to the axis A. Extending from the surface 64 and along the axis A is the molding attachment member 24 that is integral with the segment 18. The member 24 is substantially similar to the previously described member 22, the member 24 having an outer curved surface 66 that is concave, circular in cross-section and extends from the surface 64 to an end surface 68 that is substantially perpendicular to the axis A. Formed in the surface 66 are a plurality of through bores 70. In the illustrated embodiment there are two through bores 70 that form a substantially hollow area within the member 24 to flow receive and provide for set-up and adherence to the plastic core 8. The plastic core is molded adjacent to the surface 64 and thus may adhere thereto as well as to the surface 68.

With particular reference to FIGS. 2, 3 and 6, the washer 10 is annular and substantially flat, having an outer cylindrical surface 72, an inner cylindrical surface 73 and opposed planar surfaces 74 and 75 operatively disposed perpendicular to the axis A. The outer cylindrical surface 72 has a diameter the same or substantially the same as the outer diameter of the buttress plate 21 surface 44 and the spacer 9 outer surface 55 diameter. The inner cylindrical surface 73 has a diameter that is the same or substantially the same as the diameter of the inner surface 56 of the spacer 9. The inner cylindrical surface 73 is sized and shaped to receive the segment 18 threaded portion 23 and abut against the spacer 9 surface 58 near the end surface 64.

With particular reference to FIGS. 2, 3, and 7-9, the compression/distraction nut 12 has a faceted outer surface 78 hexagonal in cross-section suitable for engagement with a manipulation and tightening tool (not shown) having a wrench or socket driving feature. The nut further includes a substantially planar abutment surface 80 sized and shaped to abut and engage the washer surface 75. The nut 12 is annular and thus further includes an internal substantially cylindrical threaded surface 82 sized and shaped to mate with the threaded portion 23 of the segment 18 under rotation. The inner threaded surface 82 defines a bore with a circular cross section, the bore extending through the nut 12. Opposite the planar abutment surface 80 is a substantially planar annular rim 84 and a concave surface 86, the surface 86 extending from the faceted surface 78 to the rim 84; the rim 84 extending from the concave surface 86 to the inner threaded surface 82. In the illustrated embodiment, the nut 12 further includes a pair of opposed tooling grooves 88 formed in the rim 84 and the concave surface 86 and extending through both the outer surface 78 and the inner threaded surface 82. The grooves 88 provide further structure for a nut manipulation tool as well as access to portions of the threaded segment 23 after the nut 12 is in a final tightened position, allowing for a tool (not shown) to be inserted into one or each of the grooves 88 to deform a portion of the thread of the threaded segment 23 and thus fix or lock the nut 12 in a desired position by preventing further rotation of the threaded surface 82 with respect to the threaded segment 23. It is foreseen that a compression member other than the nut 12 may be used according to the invention, such as, for example, a compression member that is ratcheted or otherwise fixed against the spacer 9.

The core 8, spacer 9 and segments 16 and 18 may be sized and made from such materials as to provide for relatively more or less rigidity along the entire assembly 1, for example with respect to flex or bendability along the assembly 1. Such flexibility therefore may be varied by changing the outer diameter of the various sections of the core 8 and the sections 16 and 18. Also, since the distance between the bone screw assembly receivers or heads can vary, the sections 16 and 18 may need to be more or less stiff.

With reference to FIG. 10, the closure structure 27 can be any of a variety of different types of closure structures for use in conjunction with the present invention with suitable mating structure on the interior surface of the upstanding arms 35 of the receiver 31. The illustrated closure structure 27 is rotatable between the spaced arms 35, but could be a slide-in closure structure. As described above, the illustrated closure structure 27 is substantially cylindrical and includes an outer helically wound guide and advancement structure in the form of a flange form 90 that operably joins with the guide and advancement structure disposed on the interior of the arms 35. The illustrated closure structure 27 includes a lower or bottom surface 92 that is substantially planar and may include a point and/or a rim protruding therefrom for engaging the section 16 or 18 outer cylindrical surface. The closure structure 27 has a top surface 94 with an internal drive feature 96, that may be, for example, a star-shaped drive aperture sold under the trademark TORX. A driving tool (not shown) sized and shaped for engagement with the internal drive feature 96 is used for both rotatable engagement and, if needed, disengagement of the closure 27 from the arms 35. The tool engagement structure 96 may take a variety of forms and may include, but is not limited to, a hex shape or other features or apertures, such as slotted, tri-wing, spanner, two or more apertures of various shapes, and the like. It is also foreseen that the closure structure 27 may alternatively include a break-off head designed to allow such a head to break from a base of the closure at a preselected torque, for example, 70 to 140 inch pounds. Such a closure structure would also include a base having an internal drive to be used for closure removal.

In use, at least two bone screws 25 are implanted into vertebrae for use with the longitudinal connecting member assembly 1. Each vertebra may be pre-drilled to minimize stressing the bone. Furthermore, when a cannulated bone screw shank is utilized, each vertebra will have a guide wire or pin (not shown) inserted therein that is shaped for the bone screw cannula of the bone screw shank 30 and provides a guide for the placement and angle of the shank 30 with respect to the cooperating vertebra. A further tap hole may be made and the shank 30 is then driven into the vertebra by rotation of a driving tool (not shown) that engages a driving feature at or near a top of the shank 30. It is foreseen that the screws 25 and the longitudinal connecting member 1 can be inserted in a percutaneous or minimally invasive surgical manner.

With particular reference to FIGS. 1-3, the longitudinal connecting member assembly 1 is assembled by fabricating the core in the presence of the sections 16 and 18. Specifically, the core is molded to form a substantially solid cylinder between the plate surface 48 of the section 16 and the end surface 64 of the section 18, with the molding attachment members 22 and 24 being in spaced relation such that plastic flows in and about the members 22 and 24 and thereafter sets up between the surface 52 and the surface 68 and also in the bores 54 and 70, as shown in FIG. 2. After the core 8 is molded, the segment 16, the core 8 and the segment 18 form a discrete elongate connecting member, the core 8 permanently attaching the segment 16 to the segment 18, with the end 52 of the molding attachment member 22 being spaced from the end 68 of the molding attachment member 24. The core 8 is flexible and thus can bend in any direction with respect to the axis A as well as being stretchable and compressible. Thus, the segments 16 and 18 may be pulled away from one another and pushed toward one another along the axis A.

The spacer 9, the washer 10 and the nut 12 are inserted on the segment 18 at the end 62 with the nut surface 80 facing toward the end 62. The spacer 9 is moved into position over the core 8, followed by the washer 10 being moved into position near the end surface 64 and abutting against the spacer 9. The nut 12 is moved toward the threaded portion 23 and at such portion the nut 12 is rotated mating the inner threaded surface 82 with the thread of the threaded portion or segment 23. Using a tool (not shown) that engages the surface 78, the nut 12 is rotated and tightened against the washer 10 that in turn places compressive force on the spacer 9 at the surface 58. The washer 10 presses against the spacer 9 as the nut 12 is rotated, the nut 12 eventually pulling the attachment member 24 in a direction away from the member 22, placing axial tension on the core 8. The core 8 is now dynamically loaded, being in tension along the axis A while at the same time, the spacer 9 is in axial compression between the buttress plate 21 and the washer 10. Then a tool (not shown) may be used to deform the threaded segment 23 exposed at the grooves 88 to lock the nut 12 in place and thus provide an assembly 1 for implanting that is pre-loaded both in tension and compression. It is noted that viscoelastic properties of the polymers of the elastic core 8 and the elastic spacer 9 may result in material creep that may ultimately reduce overall assembly tension and stiffness. After installing the tension nut 12, completed assemblies may be allowed to rest until tension changes due to creep are minimal. The tension nut may then be torqued to calibrate overall assembly stiffness to a desired value at which point crimping through the tension nut may be performed.

With reference to FIG. 10, the pre-loaded connecting member assembly 1 is eventually positioned in an open or percutaneous manner in cooperation with the at least two bone screws 25 with the core 8, the spacer 9, the washer 10 and the nut 12 disposed between and spaced from the two bone screws 25 and with the segments 16 and 18 each being within a U-shaped channel of a cooperating bone screw 25. A closure structure 27 is then inserted into and advanced between the arms 35 of each of the bone screws 25. The closure structure 27 is rotated, using a tool (not shown) engaged with the inner drive 96 until a selected pressure is reached at which point the section 16 or 18 is urged toward, but not completely seated in the u-shaped channel of the bone screw 25. For example, about 80 to about 120 inch pounds pressure may be required for fixing the bone screw shank 30 with respect to the receiver 31 at a desired angle of articulation.

The assembly 1 is thus substantially dynamically loaded and oriented relative to the cooperating vertebra, providing relief (e.g., shock absorption) and protected movement with respect to flexion, extension, distraction and compressive forces placed on the assembly 1 and the two connected bone screws 25. The core 8 and the spacer 9 allow for some twisting or turning, providing some relief for torsional stresses. Furthermore, the compressed spacer 9 places some limits on torsional movement as well as bending movement, to provide spinal support. Furthermore, the pre-loaded core 8 (in tension) and spacer 9 (in compression) allow for both protected extension and compression of the assembly 1 located between the two bone screws 25, e.g., shock absorption.

If removal of the assembly 1 from any of the bone screw assemblies 25 is necessary, or if it is desired to release the assembly 1 at a particular location, disassembly is accomplished by using the driving tool (not shown) with a driving formation cooperating with the closure structure 27 internal drive 96 to rotate and remove the closure structure 27 from the receiver 31. Disassembly is then accomplished in reverse order to the procedure described previously herein for assembly.

Eventually, if the spine requires more rigid support, the connecting member assembly 1 according to the invention may be removed and replaced with another longitudinal connecting member, such as a solid rod, having the same diameter as the sections 16 and 18, utilizing the same receivers 31 and the same or similar closure structures 27. Alternatively, if less support is eventually required, a less rigid, more flexible assembly, for example, an assembly 1 having a core made of a more flexible material, but with end portions having the same diameter as the rigid segments 16 and 18, may replace the assembly 1, also utilizing the same bone screws 25.

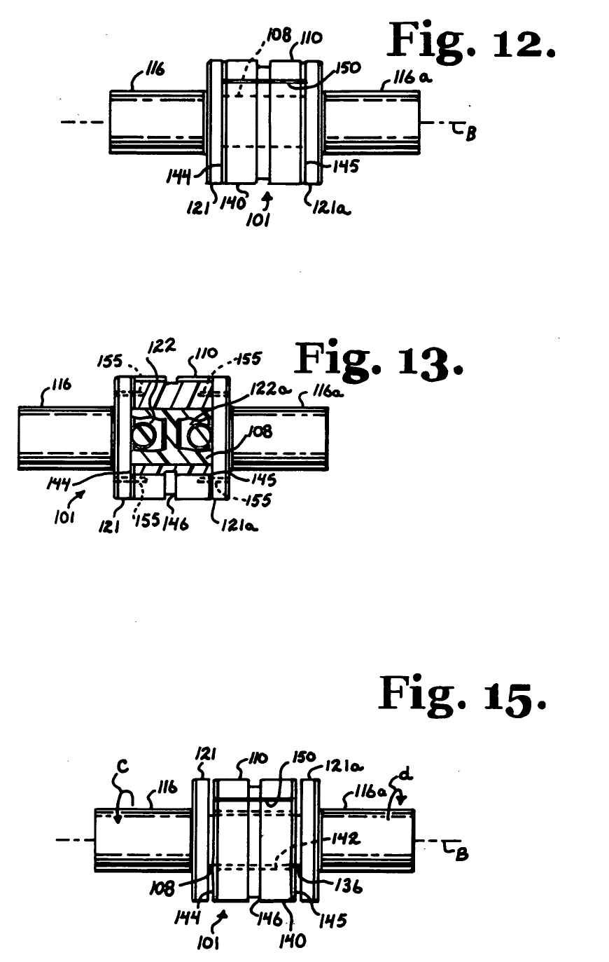

With reference to FIGS. 11-15, an alternative longitudinal connecting member assembly according to the invention, generally 101 that has a central axis B and includes a molded elastic inner core 108, an outer spacer 110 and a pair of rigid segments 116 and 116a. The segments 116 and 116a are the same or substantially similar to the segment 16 described previously herein with respect to the assembly 1. Thus, they each include a respective integral buttress plate 121 and 121a, and a respective integral molding attachment member 122 and 122a the same or similar to the plate 21 and the attachment member 22 previously described herein with respect to the segment 16. The sections 116 and 116a are placed in a mold with the members 122 and 122a in alignment along the axis B and facing one another and the core 108 that is made from the same or substantially similar material as the core 8 is molded in a manner the same or substantially similar to the core 8 with the exception that the core 108 is in the form of a cylinder having an outer surface 136 with a diameter that is smaller than an outer diameter of the buttress plates 121 and 121a. In the illustrated embodiment, the core 108 diameter is shown as being slightly smaller than a diameter of the rigid sections 116 and 116a that are substantially, uniformly cylindrical with a circular cross-section. Each of the segments 116, 116a and the core 108 sharing the same central axis B. During fabrication, the core 108 is molded so that plastic flows through apertures or bores in the members 122 and 122a and also flows around and between the members 122 and 122a, adhering to such members 122 and 122a as well as to a central or inner portion of the buttress plates 121 and 121a located near and about the axis B as best shown in FIGS. 13 and 15.

With particular reference to FIGS. 13-15, the sleeve or spacer 110 advantageously cooperates with the core 108, providing limitation and protection of movement of the core 108. The spacer 110 is substantially cylindrical and made from a plastic, such as a thermoplastic elastomer. The spacer 110 has an external substantially cylindrical surface 140 and an internal substantially cylindrical and smooth surface 142 defining a bore with a circular cross section extending through the spacer 110. The surfaces 140 and 142 extend between a pair of substantially planar end surfaces 144 and 145. When cooperating with the core 108 the end surfaces 144 and 145 are substantially perpendicular to the axis B. It is foreseen that in some embodiments, the spacer may be of square, rectangular or other cross-section including curved or polygonal shapes. In the illustrated embodiment, the spacer 110 further includes a compression groove 146. Spacers according to the invention may include one, none or any desired number of grooves. The illustrated groove 146 is substantially uniform and circular in cross-section, being formed in the external surface 140 and extending radially toward the internal surface 142. The internal surface 142 is of a slightly greater diameter than the outer diameter of the core 108 surface 136. The size of the internal surface 142 allows for axially directed sliding movement of the spacer 110 with respect to the core 108. The spacer 110 further includes a radially directed elongate slit or gap opening 150 extending therethrough between the outer surface 140 and the inner surface 142 and through the surfaces 144 and 145. The slit or gap 150 allows for opening the spacer 110 and placing the spacer 110 onto the core 108 with the gap or slit 150 widening or expanding to receive the core 108 and then elastically returning the spacer 110 to an original cylindrical shape as shown in FIG. 14, but now positioned with the inner cylindrical surface 142 in sliding, rotating engagement with the outer surface 136 of the core 108 as shown in FIG. 15. Also, as shown in FIG. 15, when the spacer 110 is initially placed on the core 108, the spacer 110 completely surrounds the core 108 with the exception that the end surfaces 144 and 145 are spaced from the buttress plates 121 and 121a. Thus a relatively small axial length of the core 108 is not initially surrounded by the spacer 110.

Prior to use with a pair of bone screws similar to that shown in FIG. 10 with respect to the assembly 1, in order to pre-compress the spacer 110 and also to pre-tension the inner core 108, the sections 116 and 116a are rotated or turned in opposite directions as illustrated by the arrows c and d in FIG. 15. Rotating the sections 116 and 116a in opposite directions twists the core 108, thereby shortening the core 108 along the axis B. Such twisting and shortening draws the buttress plate 121 toward the buttress plate 121a and compresses the spacer 110 in an axial direction. With reference to FIG. 13, once the spacer 110 is compressed by and between the plates 121 and 121a, pins 155 or other fixing devices extending through the plate 121 or 121a and the spacer 110 are inserted to fix the spacer 110 with respect to the buttress plates 121 and 121a and thus fix the core 108 in a desired twisted tensioned pre-loaded position within the spacer 110 as well as fixing the spacer 110 in pre-loaded axial compression between the plates 121 and 121a. It is foreseen, that in certain embodiments according to the invention, an outer ring or rings may be placed about the spacer 110 and fixed, such as by a spot weld, in order to retain the spacer 110 in a cylindrical shape and not have a buckle or a gap at the slit 150. If for example, the core 108, spacer 110 and rigid segments 116 and 116a are all made from a plastic that is radiolucent, such a ring, as well as the pins 155 may advantageously be made from a metal to provide a radiology marker.

In order to reduce the production of micro wear debris, that in turn may cause inflammation, it may be desirable to make the inner core 108 from a different material than the spacer 110. Additionally or alternatively, in order to result in adequate hardness and low or no wear debris, the spacer 110 inner surfaces and/or cooperating core 108 outer surfaces may be coated with an ultra thin, ultra hard, ultra slick and ultra smooth coating, such as may be obtained from ion bonding techniques and/or other gas or chemical treatments.

The assembly 101 may then be inserted between a pair of implanted bone screws 25 as illustrated in FIG. 10 with respect to the assembly 1, with the spacer 110 being disposed between the two bone screws 25.

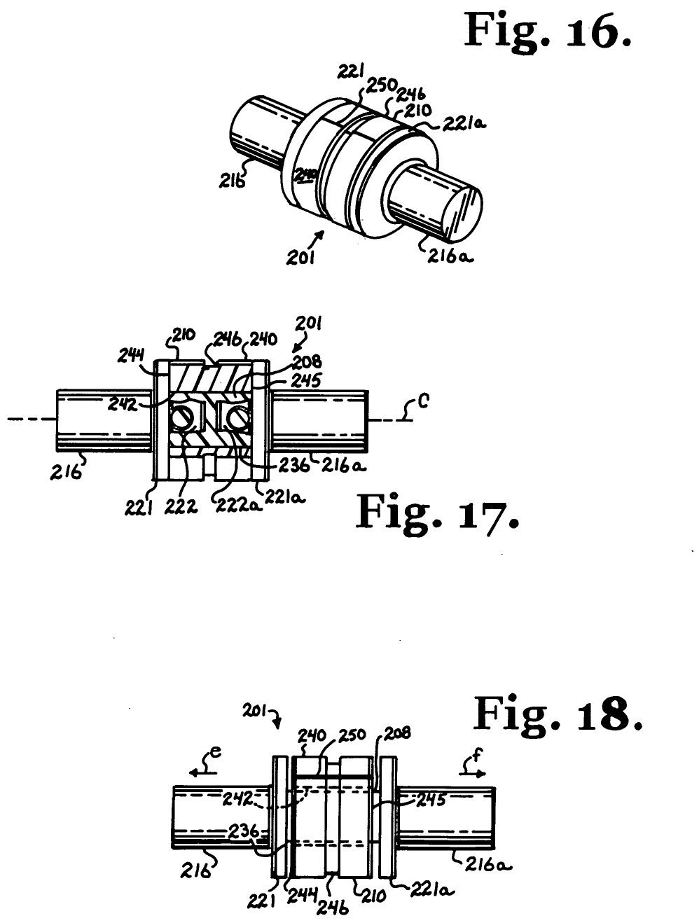

With reference to FIGS. 16-18, another alternative longitudinal connecting member assembly according to the invention, generally 201 that has a central axis C and includes a molded elastic inner core 208, an outer spacer 210 and a pair of rigid segments 216 and 216a. The inner core 208, the spacer 210 and the segments 216 and 216a are the same or substantially similar to the respective core 108, the slitted spacer 110 and the segments 116 and 116a described previously herein with respect to the assembly 101. Thus, they each include respective integral buttress plates 221 and 221a, and respective integral molding attachment members 222 and 222a the same or similar to the respective plates 121 and 121a and the attachment members 122 and 122a previously described herein with respect to the segment 116 and 116a. The sections 216 and 216a are placed in a mold with the members 222 and 222a in alignment along the axis C and facing one another and the core 208 that is made from the same or substantially similar material as the cores 8 and 108 is molded in a manner the same or substantially similar to the cores 8 and 108 as described previously herein. In the illustrated embodiment, the core 208 diameter is shown as being slightly less than the diameter of the rigid sections 216 and 216a that are substantially, uniformly cylindrical with a circular cross-section. Each of the segments 216, 216a and the core 208 shares the same central axis C. During fabrication, the core 208 is molded so that plastic flows through apertures or bores in the members 222 and 222a and also flows around and between the members 222 and 222a, adhering to such members 222 and 222a as well as to a central or inner portion of the buttress plates 221 and 221a located near and about the axis C as best shown in FIGS. 17 and 18. The core 208 differs from the core 108 previously described herein in that the core 208 has a length measured along the axis C that is shorter than an axial length of the cooperating spacer 210, the core 208 being stretched during assembly with the spacer 210 as will be described in greater detail below.

With particular reference to FIG. 18, the slitted sleeve or spacer 210 advantageously cooperates with the core 208, providing limitation and protection of movement of the core 208. The spacer 210 is substantially cylindrical and made from a plastic, such as a thermoplastic elastomer. The spacer 210 has an external substantially cylindrical surface 240 and an internal substantially cylindrical and smooth surface 242 defining a bore with a circular cross section extending through the spacer 210. The surfaces 240 and 242 extend between a pair of substantially planar end surfaces 244 and 245. When cooperating with the core 208 the end surfaces 244 and 245 are substantially perpendicular to the axis C. It is foreseen that in some embodiments, the spacer may be of square, rectangular or other cross-section including curved or polygonal shapes. In the illustrated embodiment, the spacer 210 further includes a compression groove 246. Spacers according to the invention may include one, none or any desired number of grooves. The illustrated groove 246 is substantially uniform and circular in cross-section, being formed in the external surface 240 and extending radially toward the internal surface 242. The internal surface 242 is of a slightly greater diameter than the outer diameter of the core 208 surface 236. The size of the internal surface 242 allows for sliding and rotating movement of the spacer 210 with respect to the core 208. The spacer 210 further includes a radially directed elongate slit or gap opening 250 extending therethrough between the outer surface 240 and the inner surface 242 and through the surfaces 244 and 245. The slit or gap 250 allows for opening the spacer 210 and placing the spacer 210 onto the core 208 with the gap or slit 250 widening or expanding to receive the core 208 and then elastically returning the spacer 210 to an original cylindrical shape, but now positioned with the inner cylindrical surface 242 in sliding, rotating engagement with an outer surface 236 of the core 208 as shown in FIG. 18. Also, as shown in FIG. 18, when the spacer 210 is initially placed on the core 208, the core must be stretched so that the spacer 210 fits about and completely surrounds the core 108 between the buttress plates 221 and 221a.

Prior to use with a pair of bone screws similar to that shown in FIG. 10 with respect to the assembly 1, in order to pre-compress the spacer 210 and also to pre-tension the inner core 208, the sections 116 and 116a are pulled apart utilizing a jig (not shown) in axial opposite directions as illustrated by the arrows e and f in FIG. 18. Once the elastic core 208 is lengthened a desired amount so as to receive the spacer 210 between the buttress plates 221 and 221a, the spacer 210 is inserted on the core 208 by opening or expanding the spacer 210 at the slit 250 and placing the inner spacer surface 242 about the core surface 236 with the spacer end surfaces 244 and 245 adjacent the planar surfaces of the respective buttress plates 221 and 221a. After the spacer 210 is completely disposed about the core 208 and has returned to the original cylindrical shape, the jig is then released. The elastic core 208, returning to near an original shape thereof, draws the buttress plates 221 and 221a into contact with the spacer surfaces 244 and 245, thereby placing the spacer 210 in axial compression. The spacer 210 is sized and shaped so that the elastic core 208 does not return to an original position, but is rather slightly lengthened and thus under tension. It is foreseen, that in certain embodiments according to the invention, an outer ring or rings may be placed about the spacer 210 and fixed, such as by a spot weld, in order to retain the spacer 210 in a cylindrical shape and not have a buckle or a gap at the slit 250. If for example, the core 208, spacer 210 and rigid segments 216 and 216a are all made from a plastic that is radiolucent, such a ring may advantageously be made from a metal to provide a radiology marker.

In order to reduce the production of micro wear debris, that in turn may cause inflammation, it may be desirable to make the inner core 208 from a different material than the spacer 210. Additionally or alternatively, in order to result in adequate hardness and low or no wear debris, the spacer 210 inner surfaces and/or cooperating core 208 outer surfaces may be coated with an ultra thin, ultra hard, ultra slick and ultra smooth coating, such as may be obtained from ion bonding techniques and/or other gas or chemical treatments.

The assembly 201 may then be inserted between a pair of implanted bone screws 25 as illustrated in FIG. 10 with respect to the assembly 1, with the spacer 210 being disposed between the two bone screws 25.

In the illustrated embodiments, the segments 16, 18, 116, 116a, 216 and 216a have been shown as relatively short in length, each cooperating with a single bone anchor. However, it is foreseen that in certain embodiments according to the invention such solid rod lengths may be longer to accommodate more bone anchors an thus extend along a greater length of the spine. Furthermore, it is foreseen that dynamic connecting assemblies according to the invention may include a greater number of cores 8, 108 and/or 208 and spacer combinations, each core being disposed between cooperating adjacent bone anchors.

It is also foreseen that according to the invention a core may be molded between and with two rigid members with a pre-molded spacer disposed about such core during the molding process. Thereafter, the core may be twisted and the spacer pinned in place as described above with respect to the assembly 101 to stress the core and compress the spacer. In other embodiments, the core may be stretched in a jig as described with respect to the assembly 201 and clips may be placed between the spacer and the rigid members. The clips are sized and shaped such that once released from the jig, the core contracts, placing the spacer in compression but maintaining some tension on the core.

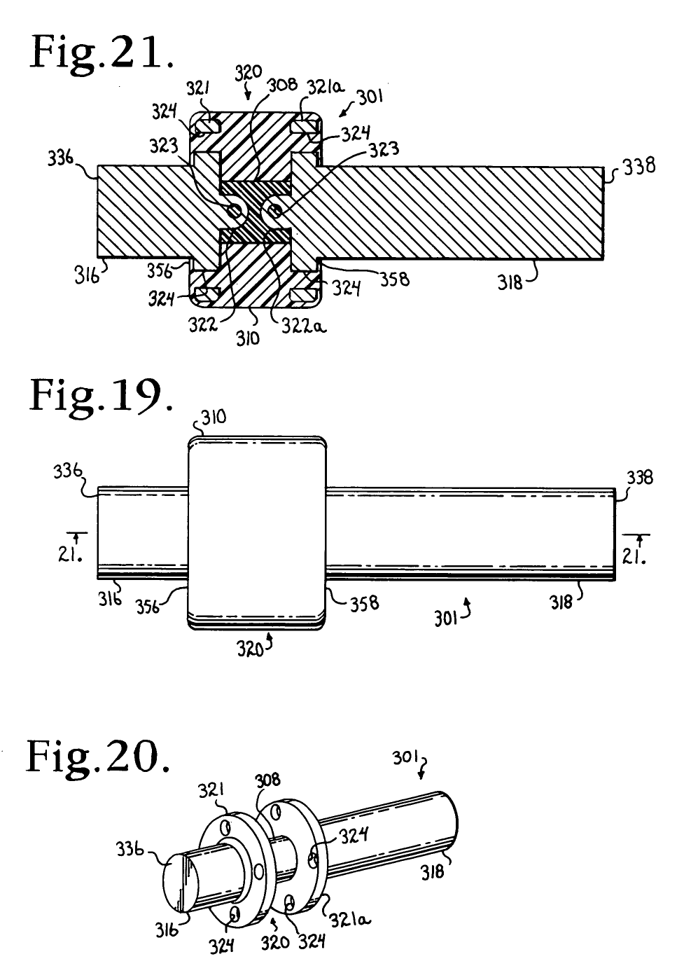

With reference to FIGS. 19-21, another alternative longitudinal connecting member assembly according to the invention, generally 301 includes an inner elastic molded core 308 cooperating with an over-molded, external or outer elastic spacer 310, resulting in a flexible and yet protected, dynamic mid-portion, generally 320. Both the elastic inner core 308 and the elastic spacer 310 may be made of a variety of elastomeric materials, the same or similar to what was described previously with respect to the elastic core 8 and the spacer 9 of the assembly 1. The core 308 and the spacer 310 may be of the same or different hardness or elasticity that may be measured, for example, in durometers. Although the illustrated core 308 is shown having an outer diameter smaller than outer diameters of cooperating rigid portions 316 and 318, the core 308 may be of a variety of diameters or widths, providing for more or less flexibility with reference to and in cooperation with the spacer 310. The core 308 is substantially similar to the core 8 previously described herein. The assembly 301 however, does not include a threaded portion, but rather a second integral plate, similar to the plates 121 and 121a of the assembly 101 previously described herein. Thus the assembly 301 includes a molded core 308 extending between the first rigid end portion 316 and the second rigid end portion 318 that are the same or similar to the respective rigid end portions 116 and 116a of the assembly 101. The assembly 301 further includes opposed stop plates 321 and 321a that are the same or similar to the respective plates 121 and 121a of the assembly 101. Opposed and facing molding attachment members 322 and 322a extending from the respective plates 321 and 321a are substantially similar to the respective attachment members 122 and 122a of the assembly 101 with the exception that both the members 322 and 322a are rounded or domed shaped as compared to the substantially planar facing surfaces of the members 122 and 122a. Such rounded surfaces provide for additional clearance between the members 322 and 322a when the core 308 is compressed and/or bent before or during operation. Similar to the apertures 54 of the assembly 1, each of the attachment members 322 and 322a further include at least one and up to a plurality of apertures 323 for receiving the elastomeric material of the core 308 there-through. Each of the stop plates 321 and 321a may be solid or include one or up to a plurality of through bores 324, illustrated as running parallel with the core 308, but not limited to a parallel configuration. The illustrated embodiment includes four bores 324 running through each plate 321 and 321a.

The solid rod portion 316 terminates at a first end 336 and is adjacent and integral to the plate 321. The solid rod portion 318 is integral with the plate 321a and terminates at an end 338 opposite the end 336. Similar to the assembly 1 and thus as illustrated in FIG. 10, each of the rod portions 316 and 318 is sized and shaped to cooperate with bone screws 25, for example. As with the assembly 1, the assembly 301 readily cooperates with a wide variety of bone anchors and closures, also as previously described herein.

With particular reference to FIG. 21, the core 308 is molded, with the elastomer flowing about the members 322 and 322a and through the apertures 323, connecting the rod portions 316 and 318 as previously described herein with respect to the cores 8 and 108 of the respective assemblies 1 and 101. Thereafter, the over-molded elastic spacer or portion 310 is molded about and in some cases adhered to the plates 321 and 321a, starting at a location 356 adjacent to or adhered to the end portion 316 and ending at a location 358 adjacent to or adhered to the end portion 318. The locations 356 and 358 are spaced from the respective plates 321 and 321a and thus the polymer of the spacer 310 completely surrounds the plates 321 and 321a and the entire molded inner core 308. An outer diameter of the over-molded spacer 310 is greater than outer diameters of the plates 321 and 321a. The core 308 may be sheathed or otherwise treated prior to over-molding of the spacer 310 so that the surface of the core 308 slidingly engages the spacer 310. It is foreseen that according to other embodiments of the invention, the plates 321 and 321a, the elastic core 308 and the over-molded spacer 310 may be of relatively constant cross-section or may have other cross-sectional geometries, including but not limited to oval, square, rectangular and other polygonal shapes. Mixtures of cross-section may be utilized, for example, the plates 321 and 321a and the spacer 310 may be substantially cylindrical while the inner core 308 may be of square or rectangular cross-section.

The longitudinal connector 301 is formed in a factory setting with the inner core 308 being held in a desired orientation by a jig, for example, attached to the rigid end portions 316 and 318. Such desired orientation of the core 308 may be a neutral state without tension; or a loaded state, such as being pulled into tension or distraction, being compressed, or being bent wherein at least a portion of the core 308 is in tension and at least a portion of the core is in compression. The jig and cooperating end portions 316 and 318 hold the core 308 in the desired neutral or loaded orientation as an elastomeric polymer is molded about the core 308 and also molded about at least a portion of the plates 321 and 321a. In the illustrated embodiment, the polymer also flows through all of the through bores 324, firmly attaching the resulting spacer 310 to the plates 321 and 321a. In some cases, the polymer is further firmly adhered to the plates 321 and 321a, occurring for example, by chemical bonding or with the aid of an adhesive. The resulting molded spacer 310 surrounds all surfaces of the plates 321 and 321a and the elastic core 308. According to some embodiments of the invention, an inner core 308 of a first durometer is first molded between the plates 321 and 321a, followed by molding of an elastic spacer 310 about the core 308 and the plates 321 and 321a, the spacer 310 exhibiting a durometer that is different (either harder or more elastic) than the durometer of the core 308. In other embodiments of the invention, the same durometer elastic material is used for both the core 308 and the spacer 310, with the core 308 being tensioned, bent or neutral during the molding of the spacer 310.

As indicated above, the connecting member assembly 301 is sized and shaped to attach to at least two bone screw assemblies to provide dynamic stabilization between such bone screws. It is noted that each of the portions 316 and 318 may also be elongate for cooperating with additional bone screws 25. In use, the assembly 301 is implanted in a manner substantially similar to that previously described herein with respect to the assembly 1. Furthermore, it is foreseen that dynamic connecting assemblies according to the invention may pre-bent and/or include a greater number of dynamic segments, each segment equipped with an over-molded spacer or a spacer cooperating with some sort of compression member for pressing the spacer against a stop or stops and tensioning or distracting an elastic core, each dynamic segment being disposed between cooperating adjacent bone anchors. The connecting assembly 301 may be substantially dynamically loaded and oriented relative to the cooperating vertebra, providing relief (e.g., shock absorption) and protected movement with respect to flexion, extension, distraction, compressive, torsion and shear forces placed on the connector 301 and the connected bone screws 25.

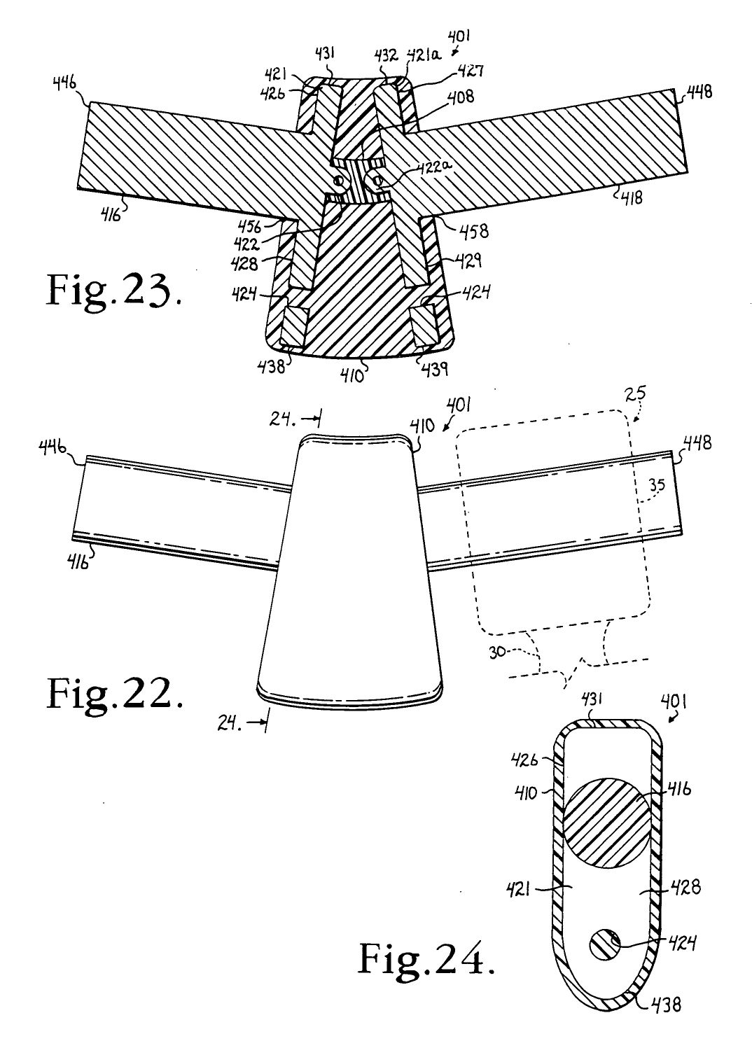

With reference to FIGS. 22-24, another alternative longitudinal connecting member assembly according to the invention, generally 401 includes an inner core 408 cooperating with an over-molded, external or outer elastic spacer 410. The core 408 and the over-molded spacer 410 may be made of materials similar to what was described previously with respect to the core 8 and spacer 10 of the assembly 1, for example. The elongate assembly 401 is substantially similar to the assembly 301 previously described herein with the exception of the geometrical design and orientation of stop plates 421 and 421a and the over-molded spacer 410 as will be described more fully below. As compared to the embodiment 301 that is shown with a neutral core 308, the embodiment 401 is shown with a partially tensioned and partially compressed core 408, with the plates 421 and 421a being rotated toward one another as will be described below.

Attached to the core 408 are a first end portion 416 and a second end portion 418, the end portion 416 being integral with a stop plate 421 and the end portion 418 being integral with a stop plate 421a. The end portions 416 and 418 are identical or substantially similar to the respective end portions 316 and 318 of the assembly 301. The stop plates 421 and 421a are substantially similar to the respective stop plates 321 and 321a with the exception of their shape and location of a through bore 424 that is similar to the bores 324 of the plates 321 and 321a. Opposed and facing molding attachment members 422 and 422a extending from the respective plates 421 and 421a are substantially similar to the respective attachment members 322 and 322a of the assembly 301. As previously described herein with respect to the assembly 301 and also illustrated in FIG. 23, rounded outer surfaces of the molding attachment members 422 and 422a provide for additional clearance between the members 422 and 422a when the core 408 is partially compressed and bent during the over molding of the spacer 410. The core 408 is the same or substantially similar in shape and function to the core 308 previously described herein with respect to the assembly 301, the core 408 being disposed between the stop plates 421 and 421a and gripping the molding attachment members 422 and 422a by filling space in apertures thereof. As with the stop plates 321 and 321a, the stop plates 421 and 421a may be solid or include one or up to a plurality of the through bores 424. The illustrated embodiment includes one bore 424 running through each plate 421 and 421a. The plates 421 and 421a are identical to one another in size and shape, differing from the plates 321 and 321a in that the plates 421 and 421a have a curved elongate form similar to a surf- or skateboard-shape as compared to the circular cross-sectional shape of the plates 321 and 321a. The plates 421 and 421a have respective posterior portions 426 and 427 located substantially on one side of the core 408 and respective anterior portions 428 and 429 located substantially on an opposite side of the core 408 from the portions 426 and 427, the portion 426 being integral with the portion 428 and the portion 427 being integral with the portion 429. The portions 428 and 429 extend a greater length in a direction away from the core 408 than the portions 426 and 427. The portions 426 and 427 are somewhat squared-off in form having substantially flat respective posterior end surfaces 431 and 432. In certain embodiments of the invention, each of the portions 426 and 427 may include a pair of opposed notches (not shown) sized and shaped for receiving an elastic band (not shown) there around, the notches being spaced from the surfaces 431 and 432. The elastic band may be made from suitable elastomeric materials, including, but not limited to, synthetic and natural rubbers and blends thereof and other elastic materials previously described herein for the core 8 and/or the spacer 10 of the assembly 1. One through bore 424 extends through each of the portions 428 and 429 and is located near but spaced from a respective curved anterior surface 438 or 439. Also, although the illustrated core 408 is shown having an outer diameter smaller than outer diameters of cooperating rigid portions 416 and 418, the core 408 may be of a variety of diameters or widths, providing for more or less flexibility with reference to and in cooperation with the spacer 410.

The solid rod portion 416 terminates at a first end 446 and is adjacent and integral to the plate 421. The solid rod portion 418 is integral with the plate 421a and terminates at an end 448 opposite the end 446. Similar to the assembly 1 and thus as illustrated in FIG. 10, each of the rod portions 416 and 418 is sized and shaped to cooperate with bone screws 25, for example (and as shown in phantom in FIG. 22). As with the assembly 1, the assembly 401 readily cooperates with a wide variety of bone anchors and closures, also as previously described herein.

With particular reference to FIGS. 23 and 24, the over-molded elastic spacer or portion 410 is molded about and in some cases adhered to the plates 421 and 421a, starting at a location 456 adjacent to or adhered to the end portion 416 and ending at a location 458 adjacent to or adhered to the end portion 418. The locations 456 and 458 are spaced from the respective plates 421 and 421a and thus the polymer of the spacer 410 completely surrounds the plates 421 and 421a and the elastic core 408. As shown in FIGS. 23 and 24, an outer peripheral surface of the over-molded spacer 410 is greater than outer peripheries of the plates 421 and 421a at every location along the surfaces of the plates 421 and 421a. The elastic core 408 may be sheathed or otherwise treated prior to molding to prohibit polymer forming the spacer 410 from adhering to the core 408 during the over-molding process and allow the core 408 to slidingly engage the spacer 410.

The longitudinal connector 401 is formed in a factory setting with the inner core 408 being held in a desired orientation that may be neutral, compressed, tensioned or in partial tension and compression. The illustrated core 408 is shown bent in partial tension and partial compression with the plate portions 426 and 427 tilted toward one another in FIGS. 22 and 23. A jig or other holding mechanism holds the connector 401 at the end portions 416 and 418 during over molding. As the jig maintains the core 408 in the desired orientation, an elastomeric polymer is molded about the core 408 and at least a portion of the plates 421 and 421a. In the illustrated embodiment, the polymer that forms the spacer 410 flows through the through bores 424, firmly attaching the resulting trapezoidal shaped spacer 410 to the plates 421 and 421a. In some cases, the polymer is further firmly adhered to the plates 421 and 421a, occurring for example, by chemical bonding or with the aid of an adhesive. The resulting molded spacer 410 surrounds all surfaces of the plates 421 and 421a and the inner elastic core 408.

As indicated above, the connecting member assembly 401 is sized and shaped to attach to at least two bone screw assemblies to provide dynamic stabilization between such bone screws. The surf-board shape of the plates 421 and 421a and cooperating molded spacer 410 advantageously provide a transfer of an operative axis of translation of the resulting medical implant assembly from a posterior to an anterior position (for example, anterior of a facet joint, guarding against overload of such facet in compression). It is noted that each of the portions 416 and 418 may also be elongate for cooperating with additional bone screws 25. In use, the assembly 401 is implanted in a manner similar to that previously described herein with respect to the assembly 1 and in an orientation as generally shown by the bone screw 25 shown in phantom in FIG. 22, with the wider and longer portion of the spacer 420 (and the plate surfaces 438 and 439) being directed anteriorly. Furthermore, it is foreseen that other portions of the assembly 401 may be pre-bent and/or include a greater number of dynamic segments (straight or pre-bent), each segment equipped with an over-molded spacer or a spacer cooperating with some sort of compression member for pressing the spacer against a stop or stops and distracting the elastic core, each dynamic segment being disposed between cooperating adjacent bone anchors. The connecting assembly 401 is substantially dynamically loaded and oriented relative to the cooperating vertebra, providing relief (e.g., shock absorption) and protected movement with respect to flexion, extension, distraction, compressive, torsion and shear forces placed on the connector 401 and the connected bone screws 25.

It is to be understood that while certain forms of the present invention have been illustrated and described herein, it is not to be limited to the specific forms or arrangement of parts described and shown.

What is claimed and desired to be secured by Letters Patent is as follows:

1. In a medical implant assembly having at least two bone attachment structures cooperating with a longitudinal connecting member, the improvement wherein the longitudinal connecting member comprises:
a) an elastic molded inner core portion;
b) a substantially non-elastic inner core portion, the elastic core portion in gripping engagement with the non-elastic inner core portion;
c) a rigid stop plate, the elastic core portion in gripping engagement with a portion of the stop plate;
d) an outer elastic spacer covering the elastic core portion; and
e) a compression member engaged with and movable along the non-elastic inner core portion, the compression member pressing the spacer against the stop plate and pre-tensioning the elastic core.

2. The improvement of claim 1 wherein the compression member is threadably mated to the non-elastic inner core portion.

3. The improvement of claim 1 wherein the outer spacer is of a first durometer and the elastic inner core portion is of a second durometer.

4. The improvement of claim 1 wherein the outer spacer has a surface with at least one groove formed therein.

5. The improvement of claim 1 wherein the compression member further comprises a planar surface disposed adjacent the spacer.

6. In a medical implant assembly having at least two bone attachment structures cooperating with a longitudinal connecting member, the improvement wherein the longitudinal connecting member comprises:
a) an inner core having a rigid stop and a molded elastic segment, the stop having a molding attachment member with at least one aperture, the elastic segment extending through the aperture and gripping the molding attachment member;
b) an outer spacer covering the elastic segment; and
c) a compression member attached to the core pressing the spacer against the stop and tensioning the elastic segment prior to implantation of the implant assembly.

7. The improvement of claim 6 wherein the outer spacer is elastic.

8. The improvement of claim 6 wherein the outer spacer has a surface with at least one groove formed therein.

9. The improvement of claim 6 wherein the compression member is threadably mated to the inner core.

10. The improvement of claim 6 wherein the compression member further comprises a planar surface disposed adjacent the spacer.

11. The improvement of claim 6 wherein the stop is a first stop and the compression member is a second stop, the molded elastic segment being located between the first and second stops, the outer spacer being over-molded about the elastic segment and between the first and second stops, the outer spacer molded during at least one of tensioning and bending of the elastic segment.

12. The improvement of claim 11 wherein the outer spacer is over-molded about and surrounding the first and second stops.

13. In a medical implant assembly having at least two bone anchors cooperating with a longitudinal connecting member, the improvement wherein the longitudinal connecting member comprises:
a) an inner core having a first non-elastic segment, a second non-elastic segment and a molded elastic segment disposed between the first and second non-elastic segments, the molded elastic segment in gripping engagement with both the first and the second non-elastic segments;
b) a stop plate adjacent the molded elastic segment; and
c) an over-molded elastic spacer surrounding the molded elastic segment and at least a portion of the stop plate, the stop plate and the elastic spacer each extending in at least one direction lateral to the inner core an amount sufficient for the stop plate and the spacer to cooperate to substantially resist bending moment of the core.

14. The improvement of claim 13 wherein the over-molded elastic spacer is of a first durometer and the molded elastic segment is of a second durometer.

15. The improvement of claim 13 wherein the over-molded elastic spacer and the molded elastic segment are of the same durometer.

16. The improvement of claim 13 wherein the stop plate is a first stop plate and further comprising a second stop plate, the over-molded elastic spacer substantially disposed between the first stop plate and the second stop plate.

17. The improvement of claim 16 wherein the over-molded elastic spacer completely surrounds the first and second stop plates.

18. The improvement of claim 16 wherein the first and second stop plates are elongate in an anterior operational direction.

19. The improvement of claim 16 wherein the molded elastic segment is in tension during over-molding of the spacer about the elastic segment and the stop plates.

20. The improvement of claim 16 wherein the molded elastic segment is bent prior to over-molding of the elastic spacer about the elastic segment and the stop plates.
